(19) United States
(12) Patent Application Publication
(10) Pub. No.: US 2022/0296414 A1
(43) Pub. Date: Sep. 22, 2022

(54) Facilitated Kinking Fold Pads

(71) Applicant: C. R. Bard, Inc., Franklin Lakes, NJ (US)

(72) Inventors: Jacob A. Bible, Covington, GA (US); Alexandra A. Falis, Marietta, GA (US); Ronald N. Legaspi, Alpharetta, GA (US); Abigail A. Wilms, Tucker, GA (US); Sudhakar Jagannathan, Alpharetta, GA (US)

(21) Appl. No.: 17/697,879

(22) Filed: Mar. 17, 2022

Related U.S. Application Data
(60) Provisional application No. 63/162,953, filed on Mar. 18, 2021.

(51) Int. Cl. A61F 7/08; A61F 7/00

(52) U.S. Cl. CPC A61F 7/08; A61F 7/0085; A61F 2007/0024; A61F 2007/0039

(57) ABSTRACT

Disclosed herein is a system, apparatus and method directed to reducing patient contact area of a medical pad for exchanging thermal energy between a targeted temperature management (TTM) fluid and a patient. The system, apparatus and method pertain to a medical pad that includes a fluid containing layer for containing the TTM fluid and a patient contact surface. The fluid containing layer is configured for circulating the TTM fluid within the fluid containing layer. The patient contact surface defines a patient contact area to facilitate thermal energy exchange with the patient. The pad can be segmented into a main pad section and one or more foldable sections configured to be folded by a user, thereby reducing the patient contact area. The circulation of the TTM fluid within the fluid containing layer may be constricted by a fold of the one or more foldable sections while folded.

### PRIORITY

This application claims the benefit of priority to U.S. Provisional Application No. 63/162,953, filed Mar. 18, 2021, which is incorporated by reference in its entirety into this application.

### SUMMARY

Briefly summarized, embodiments disclosed herein are directed to systems, methods and apparatuses for reducing patient contact area of a medical pad for exchanging thermal energy between a targeted temperature management (TTM) fluid and a patient, that is, cooling or heating a patient to provide medical benefits, such as neuroprotection following a stroke or surgery.

One problem that often arises with TTM is applying medical pads to accommodate patients of different sizes effectively and comfortably. An ill-fitting medical pad may impede effective transmission of thermal energy between the pad and the patient, and may also give rise to patient discomfort. The disclosed embodiments of devices and methods can address this problem by adjusting the patient contact area of the thermal pad to better accommodate different patient sizes.

Disclosed herein is a medical pad for exchanging thermal energy between a TTM fluid and a patient. The medical pad can comprise a fluid containing layer for containing the TTM fluid and a patient contact surface. The fluid containing layer is configured for circulating the TTM fluid within the fluid containing layer. The patient contact surface defines a patient contact area to facilitate thermal energy exchange with the patient. The pad is segmented into a main pad section and one or more foldable sections configured to be folded by a user, thereby reducing the patient contact area.

In some embodiments, the circulation of the TTM fluid within the fluid containing layer is constricted by a fold of the one or more foldable sections while folded. In some embodiments, the TTM fluid does not circulate in the one or more foldable sections while folded. In some embodiments, the fold comprises a kink that constricts the circulation. In some embodiments, the pad is segmented by means of perforation or holes disposed through a thickness of the pad. In some embodiments, the pad is configured to be folded along the perforation or holes. In some embodiments, the thickness of the pad narrows in a vicinity of the perforation or holes.

In some embodiments, the fluid containing layer narrows within the pad in the vicinity of the perforation or holes. In some embodiments, the pad further comprises hook and loop fasteners. The hook and loop fasteners (e.g., VELCRO®) is configured to secure the one or more foldable sections to the main pad section while the one or more foldable sections are folded. In some embodiments, kinking of the pad is facilitated by a user manually applying pressure at a crease of fold thereby causing the hook and loop fasteners to engage. In some embodiments, the one or more foldable sections are separately foldable. In some embodiments, the patient contact surface conforms to skin of the patient.

In some embodiments, the medical pad further includes a filter coupled to the fluid containing layer so that the TTM fluid circulating through the fluid containing layer passes through the filter. In some embodiments, the filter comprises a porous wall disposed parallel to a continuous flow path through the filter.

Also disclosed herein is a method of providing a TTM therapy to a patient. The method comprises providing a TTM system. The TTM system comprises a TTM module configured to provide a TTM fluid, a thermal pad, and a fluid delivery line (FDL) extending between the TTM module and the thermal pad. The thermal pad is configured to receive the TTM fluid from the TTM module to facilitate thermal energy transfer between the TTM fluid and the patient. The FDL is configured to provide TTM fluid flow between the TTM module and the thermal pad. The thermal pad comprises a patient contact surface defining a patient contact area to facilitate thermal energy exchange with the patient. The pad is segmented into a main pad section and one or more foldable sections. The method further comprises applying the thermal pad to the patient. The method further comprises folding the one or more foldable sections, thereby reducing the patient contact area of the thermal pad. The method further comprises delivering TTM fluid from the TTM module to the thermal pad via the FDL.

In some embodiments, the thermal pad further comprises a fluid containing layer for containing the TTM fluid, the fluid containing layer configured for circulating the TTM fluid within the fluid containing layer. The circulation of the TTM fluid within the fluid containing layer is constricted by a fold of the one or more foldable sections while folded.

In some embodiments, the thermal pad further comprises a fluid containing layer for containing the TTM fluid, the fluid containing layer configured for circulating the TTM fluid within the fluid containing layer. The fluid containing layer narrows within the pad in the vicinity of the perforation or holes. In some embodiments, the method further comprises unfolding the one or more foldable sections.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1 illustrates a TTM system using medical pads for heating and/or cooling a patient, according to some embodiments;

FIG. 2 illustrates TTM medical pads being placed on a patient, according to some embodiments;

FIG. 3 illustrates a structure of an exemplary medical pad, according to some embodiments;

FIG. 4A shows a patient with an oversized medical pad, according to some embodiments;

FIG. 4B shows a second patient with an undersized medical pad, according to some embodiments;

FIG. 5 illustrates a medical pad with foldable extensions, according to some embodiments;

FIGS. 6A-6D illustrate exemplary cross-sectional structures of a medical pad with foldable extensions, according to some embodiments;

FIGS. 7A-7C illustrate exemplary embodiments of a medical pad including foldable extensions, according to some embodiments;

FIGS. 7D-7F illustrate user-operated mechanisms for forming a kink in a medical pad, according to some embodiments;

FIG. 7G illustrates a medical pad having an alternatively-shaped extension, according to some embodiments;

FIG. 8A shows usage of the medical pads with folded extension sections, according to some embodiments, on the patient of FIG. 4A;

FIG. 8B shows usage of the medical pads with unfolded extension sections, according to some embodiments, on the patient of FIG. 4B;

FIG. 9 shows a flowchart of a method for providing a TTM therapy to a patient, according to some embodiments;

FIG. 10A is an exploded perspective view of a TTM fluid filter, in accordance with some embodiments;

FIG. 10B is a cross-sectional side view of the filter of FIG. 6A, in accordance with some embodiments; and

FIG. 10C is a cross-sectional detail view of the thermal contact pad of FIG. 2 incorporating the filter of FIG. 6A, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a probe disclosed herein includes a portion of the probe intended to be near a clinician when the probe is used on a patient. Likewise, a “proximal length” of, for example, the probe includes a length of the probe intended to be near the clinician when the probe is used on the patient. A “proximal end” of, for example, the probe includes an end of the probe intended to be near the clinician when the probe is used on the patient. The proximal portion, the proximal end portion, or the proximal length of the probe can include the proximal end of the probe; however, the proximal portion, the proximal end portion, or the proximal length of the probe need not include the proximal end of the probe. That is, unless context suggests otherwise, the proximal portion, the proximal end portion, or the proximal length of the probe is not a terminal portion or terminal length of the probe.

With respect to “distal,” a “distal portion” or a “distal end portion” of, for example, a probe disclosed herein includes a portion of the probe intended to be near or in a patient when the probe is used on the patient. Likewise, a “distal length” of, for example, the probe includes a length of the probe intended to be near or in the patient when the probe is used on the patient. A “distal end” of, for example, the probe includes an end of the probe intended to be near or in the patient when the probe is used on the patient. The distal portion, the distal end portion, or the distal length of the probe can include the distal end of the probe; however, the distal portion, the distal end portion, or the distal length of the probe need not include the distal end of the probe. That is, unless context suggests otherwise, the distal portion, the distal end portion, or the distal length of the probe is not a terminal portion or terminal length of the probe.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

The effect of temperature variations on the human body has been well documented. Elevated temperatures may be harmful to the brain under normal conditions, and even more importantly, during periods of physical stress, such as illness or surgery. Conversely, lower body temperatures, or mild hypothermia, may offer some degree of neuroprotection. Moderate to profound hypothermia (below 32° C.) tends to be more harmful to the body and may lead to death.

Targeted Temperature Management (TTM) refers to cooling or heating a patient to provide medical benefits, such as neuroprotection following a stroke or surgery. TTM or thermoregulation can be viewed in two different ways. The first aspect of temperature management includes treating abnormal body temperatures, i.e., cooling the body from elevated temperatures (hyperthermia), or warming the body to manage hypothermia. Hypothermia may occur in response to exposure to cold environments, trauma, or long complex surgical procedures. Hyperthermia may occur in response to systemic inflammation, sepsis, stroke, or other brain injury.

The second aspect of thermoregulation is a treatment that employs techniques that physically control a patient's temperature to provide a physiological benefit, such as cooling for a degree of neuroprotection. Studies have shown that treatment with mild hypothermia, defined as lowering core body temperature 2-3° C., confers neuroprotection in stroke victims, and may hasten neurologic recovery and improve outcomes when applied for 24 to 72 hours in cases of traumatic brain injury. In particular, research suggests that brain damage from a stroke may take hours to reach maximum effect. Neurologic damage may be limited and the stroke victim's outcome improved if a neuroprotectant therapy, such as cooling, is applied within this time frame.

A TTM system using medical pads can regulate body temperature for patients who undergo procedures requiring therapeutic TTM and/or to assist in controlling temperature for specific medical or surgical conditions. Such a system is described in U.S. Pat. No. 6,645,232, filed Oct. 11, 2001, and titled “Patient Temperature Control System with Fluid Pressure Maintenance,” and the medical pads are described in U.S. Pat. No. 6,375,674, filed Jan. 3, 2000, and titled “Cooling/Heating Pad and System,” both of which are incorporated herein by reference.

One problem that often arises with targeted temperature management (TTM) systems is applying medical pads to accommodate patients of different sizes effectively and comfortably. An ill-fitting medical pad may impede effective transmission of thermal energy between the pad and the patient, while also giving rise to patient discomfort. For example, if the TTM pad is oversized, covering too much of a patient's body surface, the patient may be heated or cooled too strongly by the TTM pad. In another example, an undersized TTM pad may not heat or cool the patient sufficiently. Embodiments of the disclosed apparatus and system can address this problem.

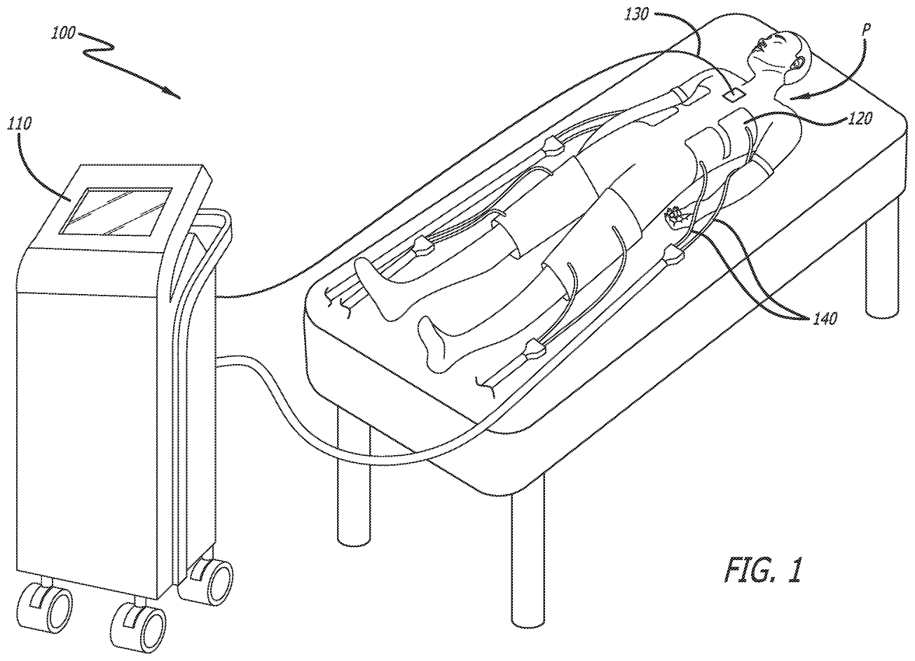

Reference is now made to FIG. 1, which illustrates a TTM system 100 using medical pads 120 for heating and/or cooling a patient P, according to some embodiments. The illustrated patient temperature control system 100 is a thermoregulatory system and apparatus that monitors and controls patient temperature within a range of 32° C. to 38.5° C. (89.6° F. to 101.3° F.). TTM system 100 is selectively interconnected to one or more medical contact pads 120 for exchanging thermal energy with patient P, and can also include a circulating pump for drawing temperature-controlled fluid (e.g., water or a gas) through pads 120 under negative pressure.

In some embodiments, TTM system 100 can include a control module 110, one or more disposable medical contact pads 120, a remote display in control module 110, a patient temperature probe 130, one or more fluid circulation lines 140, such as inlet and outlet lines to and from pads 120, and any additional accessories. In a typical embodiment, there may be two pads 120 placed on the patient's upper body as shown, and two on the patient's lower body. The TTM system 100 uses negative pressure to draw temperature-controlled fluid, such as water ranging between 4° C. and 42° C. (39.2° F. and 107.6° F.), through the pads 120 at approximately 0.7 liters per minute per pad. This results in heat exchange between the circulating fluid and the patient P. The patient temperature probe 130 is connected to the control module 110, and provides patient temperature feedback information to an internal control algorithm of control module 110. Based on such an internal control algorithm, control module 110 can increase or decrease the circulating water temperature so as to heat or cool patient P to a target patient temperature, which can be set by the clinician.

Fluid circulation lines 140 may include opposing tubing assemblies for interconnection to outlet and inlet ports of the circulating pump, with pads 120 fluidly interconnectable by means of opposing pad manifolds. FIG. 1 also illustrates the interconnection of one or more external patient temperature sensors 130 with a signal conditioning interface of control module 110. The temperature information received from external temperature sensors 130 may be utilized at a processor of control module 110 to determine the amount and rate of thermal exchange to be affected by system 100 in relation to the preset or user-defined patient target temperature. Accordingly, the processor may provide appropriate control drive signals to a heater, radiator/fan and/or auxiliary pump of TTM system 100. In an embodiment, the circulating pump, heater, radiator/fan, and/or auxiliary pump may be housed within control module 110.

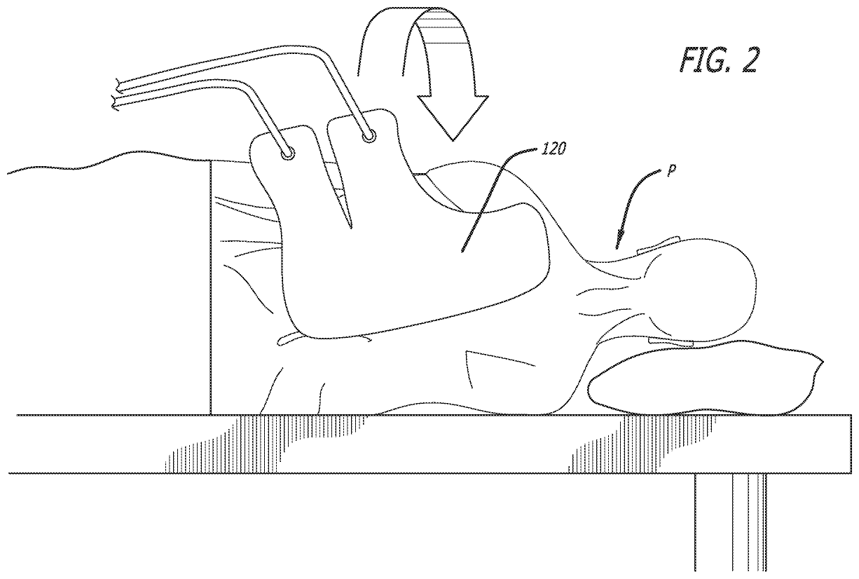

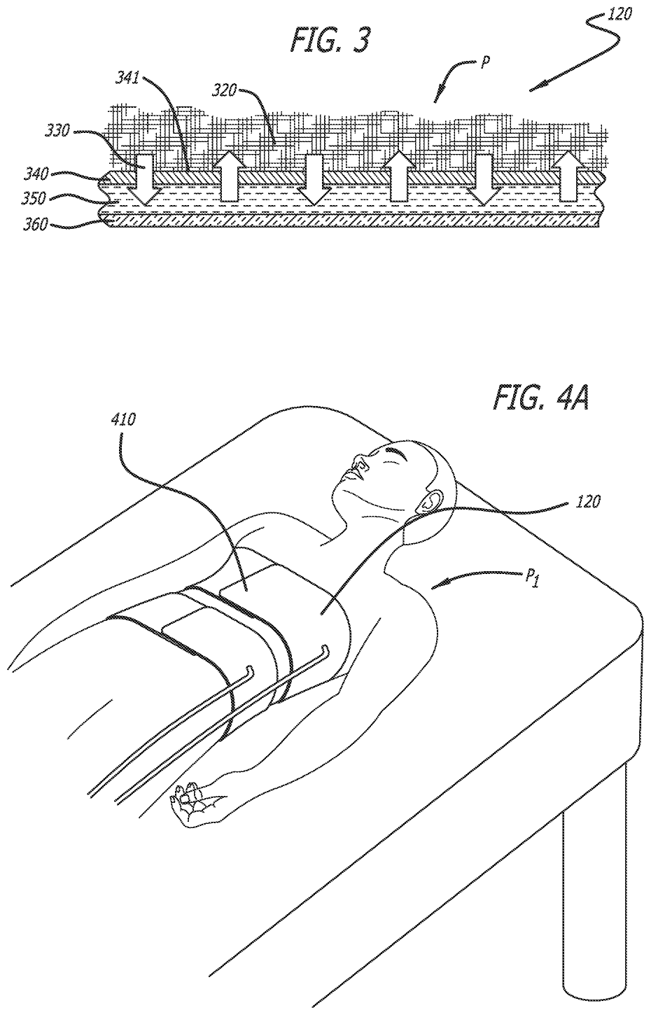

FIG. 2 illustrates a TTM medical pad 120 being placed on a patient P, according to some embodiments. Pad 120, and particularly an inner layer of pad 120 containing biocompatible hydrogel, can conform to the patient's skin, and thereby provide good thermal contact with patient P. The medical pad 120 can include several layers: an inner biocompatible hydrogel layer that adheres and conforms to the patient P, a fluid containing layer, one or more thin film layers which serve as a fluid barrier, and an outer insulating layer which prevents heat transfer to the environment (see FIG. 3). The hydrogel layer can have sufficient adhesive strength to hold pads 120 in place on patient P during the TTM therapy, yet not cause tissue damage when subsequently removed.

The pads may be available in extra-small, small, medium, and large sizes, as well as a universal pad. The clinician can determine the style, size, and number of pads 120 to be applied to patient P based on the patient procedure, application, or the available body surface area on patient P. For example, the clinician may place two pads 120 on the patient's upper body, such as on the patient's back and torso as illustrated in FIG. 2, and two pads on the patient's lower body, for example wrapped around the patient's thighs. The medical pads 120 will provide the best performance when the maximum number and correct size are used.

Due to the negative fluid pressure applied by system 100, significant fluid leakage will not occur, even if pads 120 are damaged or broken while fluid is flowing. Accordingly, pads 120 can be applied to the patient while fluid is already flowing through the pads. Depending on the objective of the treatment and the patient's level of arousal, pads 120 may be pre-warmed or pre-cooled prior to placement.

In order to place TTM pad 120, a clinician will first align the top of a first upper body pad 120 with axilla of the patient's outstretched arm. The clinician will then place the long side of pad 120 along the side of the patient's spine. Next, the clinician can wrap pad 120 from back to front as illustrated, ensuring that the pad's fluid inlet and outlet lines are lying anteriorly. For the lower body, the clinician can align the first lower body pad's lines with the knee and point downward. The clinician will wrap the long end of the first lower body pad laterally, and overlap medially if needed.

The clinician may then turn the patient P and place a second upper body pad on the patient's other side, leaving a space along the patient's spine. Next, the clinician can wrap a second lower body pad around the patient's other leg, ensuring that the shorter edge is placed medially and the longer side is wrapped laterally. Finally, if additional surface coverage is needed, the clinician can optionally place a universal medical pad on the patient's abdomen.

The medical pads 120 have inlet and outlet lines for the fluid flow, referred to herein as pad lines (see FIG. 1). These lines are connected to the pads 120 by means of a pad manifold. In particular, a Y-shaped fluid delivery line (FDL) contains one-way valves that connect to pad line connectors (e.g., a total of six connectors). Each side of the fluid delivery line can be placed by the patient's feet or along the patient's lower legs. The connectors can accommodate a full set of four pads 120 plus a maximum of two optional universal medical pads for larger patients. While holding the pad line tubing, the clinician can insert a pad line connector into the pad fluid delivery line manifold. For example, the clinician can push a respective connector toward the manifold to release associated catches, and then pull apart. Subsequently, the clinician can disconnect the lines, e.g., by squeezing wings on the connector together.

FIG. 3 illustrates a structure of an exemplary medical pad, according to some embodiments. TTM medical pad 120 comprises inner biocompatible hydrogel layer 340, which is a conformable, thermally conductive layer that can adjoin and conform to patient's skin 320. Further, the pad 120 may include an adhesive layer 341 disposed on the skin contacting side of the hydrogel layer 340 for adhering the pad 120 to the patient's skin 320. While not shown, a removable release liner may be provider over the adhesive surface 341 to protect the adhesive surface 341 from contamination while the pad 120 is not in use.

Pad 120 additionally comprises fluid containing layer 350 and insulation layer 360 for preventing loss of thermal energy to the environment. The fluid containing layer 350 can be defined between one or more film layers and/or insulation layer 360. The fluid can be heated or cooled to a temperature between 4° C. and 42° C. (39.2° F. and 107.6° F.), and can circulate through fluid containing layer 350, exchanging thermal energy 330 with patient's skin 320 via hydrogel layer 340, so as to warm or cool patient P to the target temperature. Although in this example, thermal energy 330 is shown flowing from skin 320 to the fluid in layer 350, heat 330 can flow in either direction between patient P and layer 350, so as to heat or cool patient P to the target temperature.

Alternatively, in some embodiments, pad 120 comprises hydrogel layer 340, a thin film layer which serves as a fluid barrier, and outer insulating layer 360 comprising foam with water channels.

A hydrogel is an appropriate material for layer 340 because the hydrogel is biocompatible, its adhesive strength does not tend to increase over time as compared with traditional adhesive, it tends to envelop hair on patient's skin 320, thereby facilitating good thermal contact, and its high water content results in relatively high thermal conductivity. Accordingly, hydrogel layer 340 may function as a thermally conductive layer, while also having sufficient adhesive properties so as to integrally provide an adhesive surface. Alternatively, in some embodiments, the conformable, thermally conductive layer and adhesive surface can be comprised of different materials. For example, an appropriate adhesive material may be sprayed or otherwise applied onto the surface of a layer of an appropriate conformable, thermally conductive material different than the adhesive material.

Fluid containing layer 350 can include tortuous fluid flow paths, which can be defined by dimples or other elongated members on insulation layer 360 or within the fluid containing layer 350. Such tortuous fluid flow paths can serve to regulate the fluid flow, and to inhibit the formation of boundary layers wherein some of the fluid remains substantially stationary along the inside surfaces of the fluid containing layer 350. Such boundary layers could reduce the efficiency of the pad 120 because the stationary fluid remains within the fluid containing layer 350, but eventually becomes ineffective at heating or cooling patient P as it approaches the existing temperature of patient P. Furthermore, the crisscrossed geometry of elongated members defining the tortuous flow paths also facilitates an even, low pressure drop between the inlet and the outlet required by a negative flow pressure circulating system.

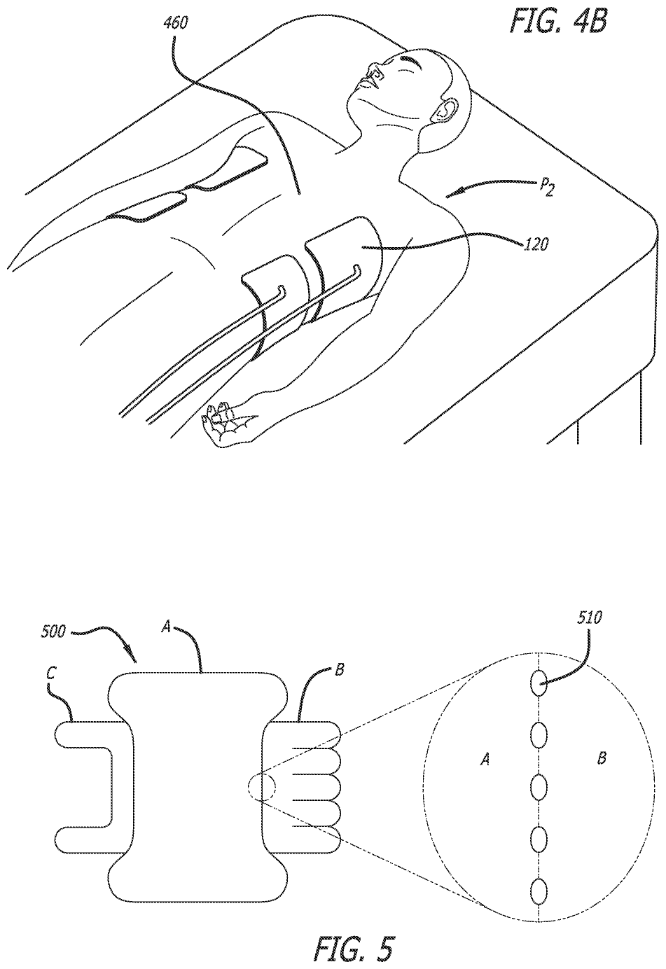

One need that frequently arises with targeted temperature management (TTM) is for the TTM medical pads to accommodate different patient sizes. An inadequately-fitting medical pad, such as those shown in FIGS. 4A-4B, may impede effective transmission of thermal energy between the pad and the patient, and may also give rise to patient discomfort. While medical pads 120 may be premanufactured in various standardized sizes (for example, five standardized sizes for adults and four sizes for infants and children), as well as in “universal” pad sizes that provide supplementary coverage, accommodating patient sizes more precisely remains a common need with TTM technology. For example, universal pads are designed to supplement coverage on arbitrary areas of a patient's body, but are not specifically designed to conform to a particular area, such as a patient's torso, back, or legs. Disclosed herein are embodiments of TTM medical pads and methods for adjusting the patient contact area to better accommodate patients of different sizes.

FIG. 4A shows a patient P₁ with an oversized medical pad 120. In this example, as pad 120 is too large for patient P₁, portions 410 of pad 120 entirely cover the chest of patient P₁, which may run counter to the intention of the clinician overseeing the TTM therapy. FIG. 4B, discussed below, illustrates utilization of an undersized medical pad.

In addition to causing patient discomfort, such a situation could lead to energy waste, as well as ineffective temperature management. Because portions 410 of pad 120 overlap one another, and do not directly contact patient P₁, some of the heating and cooling power of pad 120 goes to waste. For example, since the TTM fluid flowing through pad 120 is at a substantially uniform temperature, net heat is not expected to be exchanged between the overlapping portions of pad 120. Instead, the excess flow of TTM fluid may heat or cool the ambient air around patient P¹. In another example, because portions 410 of pad 120 cover too much body surface of patient P₁, patient P₁ may be heated or cooled too strongly by pad 120. In an acute case, overheating or overcooling patient P₁ could potentially engender a risk of medical complications, particularly if patient P₁ is in a vulnerable state, such as recovering from a stroke, from another medical emergency, or from a surgery. Thus, there is a need for a method to adjust the size of pad 120 in order to improve the fit of pad 120.

FIG. 4B shows a second patient P₂ with an undersized medical pad 120. Because patient P₂ is larger than patient P₁, a greater total flow volume of TTM fluid is needed to heat or cool patient P₂ effectively. Moreover, patient P₂ has a larger surface area than patient P₁, therefore additional pad surface area is needed to cover patient P₂ in order to heat or cool patient P₂ effectively.

In this example, portion 460 of patient P₂ is uncovered by pad 120. The situation shown in FIG. 4B can also lead to ineffective temperature management, as too little TTM fluid may flow through pad 120, and too little thermal energy may be exchanged with patient P₂, to adequately heat or cool patient P₂. In fact, undersized medical pad 120 resulting, in this example, in uncovered areas 460 on patient P₂ could pose an even greater hazard than overlapping pad portions 410 in FIG. 4A, since undersized pad 120 may fail to heat or cool patient P₂ adequately. Moreover, while universal TTM pads could be used as supplementary coverage for portion 460 of the body of patient P₂, such universal pads are not specifically designed to conform to a particular area of the patient's body, such as uncovered area 460. Thus, there is a need for a solution to adjusting the size of pad 120. Embodiments of the disclosed apparatus, system, and methods can provide such a solution by folding or unfolding extension sections on a TTM pad.

FIG. 5 illustrates a medical pad 500 with foldable extensions, according to some embodiments. In this example, pad 500 includes main pad section A and foldable extensions B and C. The foldable extensions B and C can be segmented by perforations 510, holes, a seam, or some other structure that delineates or segments pad 500. It should be understood that the shape of the pad embodiments disclosed herein is not intended to be limiting. Instead, the features of the disclosure are intended to apply to various shape pads such as those that may be particularly shaped to confirm to a patient's anatomy. In some instances, various shapes may include circles, ovals, triangles, rectangles, trapezoids, parallelograms, rhombuses, crescents, etc. It is noted that FIG. 2 illustrates a main section of the medical pad (e.g., not a extension) having a “L” shape.

In the example of FIG. 5, medical pad 500 is shown unfolded, such that extension sections B and C are substantially parallel and level with main pad section A. In this case, the TTM fluid can flow unobstructed through all three sections. Specifically, the TTM fluid can flow from main section A, which can be connected to the pad line, to extensions B and C. As a result, extension sections B and C, as well as main pad section A, can exchange thermal energy with the patient. In effect, when extensions B and C are unfolded, the pad 500 has an enlarged patient contact surface corresponding to all three sections. Accordingly, pad 500 can heat or cool the patient from this enlarged contact surface. In various embodiments, the percentage of contact area increase compared with the main pad section A may be up to about 10 percent, 25 percent, 50 percent, 100 percent, etc.

In order to reduce the contact area of pad 500, for example while treating a smaller patient, a user can fold extensions B and C along perforations 510, holes, or another seam or segmenting structure that separates the extensions from the main pad. When extensions B and C are folded, a kink forms along the perforations 510, holes, seam, or segmenting structure. Such a kink constricts fluid flow into extensions B and C, so that TTM fluid flows only in main pad section A. Accordingly, the contact area of pad 500 is effectively reduced to main section A.

In some embodiments, extensions B and C are separately foldable. For example, a user might fold section B while leaving section C unfolded. In another example, a user might fold section C while leaving section B unfolded. In a third example, there may be multiple foldable extension sections, such as four or six, and the user may fold any subset of these extension sections at any given time. In such cases, fluid flow to any folded extension sections may be constricted, whereas fluid flow may continue freely in the main section as well as any unfolded sections. As a result, the user can customize the medical pad 500 in a variety of manners, thereby providing a pad of a size and shape well-suited to the patient. Moreover, the appropriate fit of medical pad 500 reduces wasted energy, and enables the pad to heat or cool a patient efficiently and effectively.

Conversely, the user can also expand the pad 500 from a folded state by unfolding some or all of the extension sections, so that at least some of the extension sections are disposed in contact with the patient. In some embodiments, pad 500 may comprise multiple folds defining a bellows arrangement of multiple extension sections.

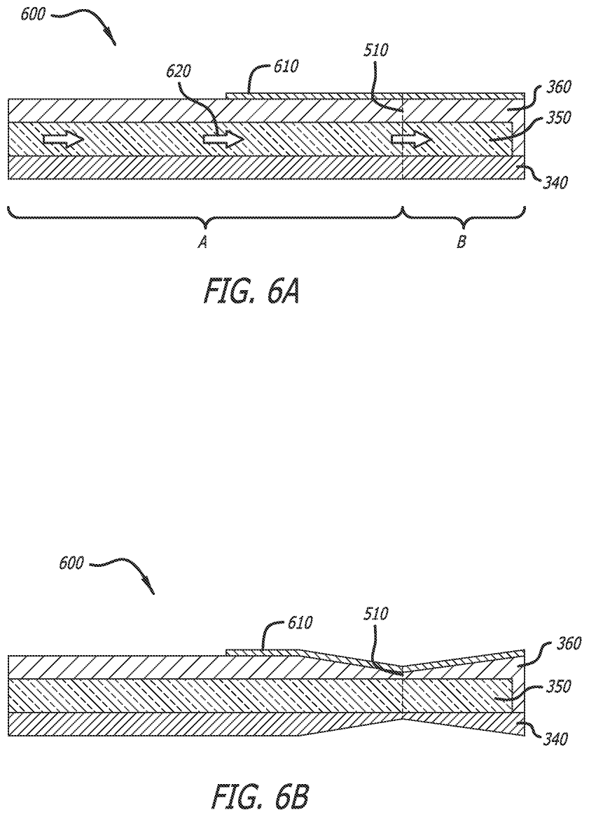

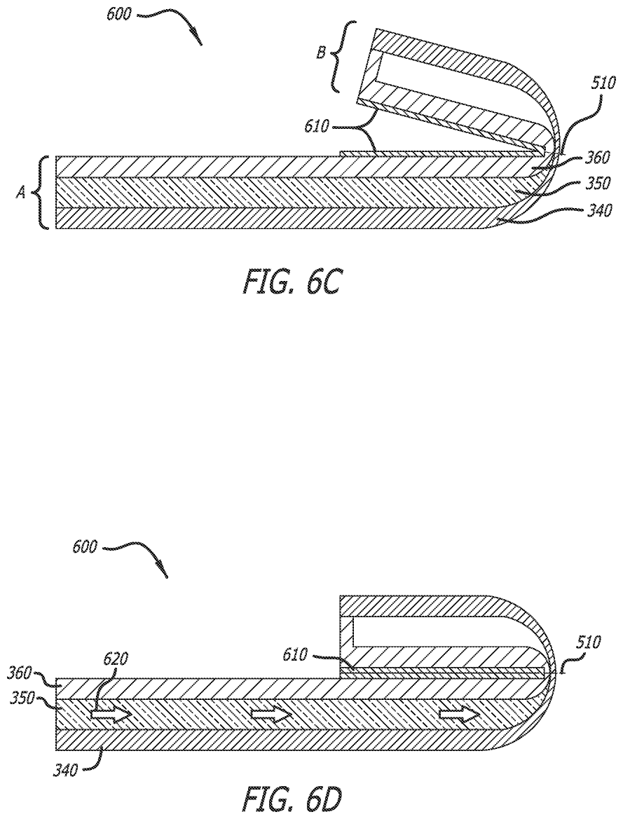

FIG. 6A illustrates an exemplary cross-sectional structure of the medical pad 500 of FIG. 5 with foldable extensions in an unfolded configuration, according to some embodiments. As in the example of FIG. 3 above, pad 500 includes inner biocompatible hydrogel layer 340, fluid containing layer 350, and insulation layer 360. In addition, foldable medical pad 500 includes perforations 510, holes, or another seam or segmenting structure, and hook and loop fasteners 610, which can be used to maintain the sections in a stable position when folded. In this example, hook and loop fasteners 610 cover both foldable extension section B of pad 500, and part of main pad section A. In some embodiments, a different method may be used to fasten the folded sections, such as adhesive, cohesion, or snap buttons, and is not limited by the present disclosure.

As shown, the TTM fluid (e.g., water or a gas) can flow 620 through the pad 500 while pad 500 is in an unfolded configuration. In particular, the fluid flows 620 from main pad section A past the region containing the perforations 510 or segmenting structure, and into extension section B.

In particular, in a typical embodiment, the fluid circulation lines 140 (see FIG. 1) can include an inlet line to the medical pad 500, and an outlet line from pad 500. Accordingly, in this example, a fluid inlet line can bring fluid flow 620 into main pad section A, while a fluid outlet line can remove the fluid flow 620 from extension section B. In another embodiment, both inlet and outlet lines can be located at a first edge of main pad section A, and the fluid flow 620 can reverse at a second edge of extension section B, flowing back to the outlet line at the first edge of section A.

Thus, in this example, the TTM fluid flows 620 throughout pad 500, with its enlarged patient contact surface corresponding to both sections A and B, with no obstruction or reduction of flow. Accordingly, both sections A and B can contribute to heating or cooling the patient.

FIG. 6B illustrates another exemplary cross-sectional structure of a medical pad 500 with perforated foldable extensions, according to some embodiments. Pad 500 again includes an inner biocompatible hydrogel layer 340, a fluid containing layer 350, and an insulation layer 360. In this example, the thickness of pad 500 narrows in the vicinity of perforations, holes, seam, or segmenting structure 510, thereby facilitating folding of pad 500. In particular, the narrowing thickness of pad 500 may make the pad easier for the user to fold along perforations, seam, or segmenting structure 510.

As shown, the individual layers of pad 500, including hydrogel layer 340 and insulation layer 360, can narrow in the vicinity of perforations 510. In some embodiments, fluid containing layer 350 may also narrow in the vicinity of perforations 510. In various embodiments, any subset or combination of these layers may narrow. In some embodiments, the narrowing of these layers may be slight (for example, a thickness of the layers may narrow by less than approximately 10%, 25%, or 50%), such that each layer can still perform its functions when in an unfolded state. In addition, hook and loop fasteners 610 can follow the narrowing contour of pad 500, as shown.

FIG. 6C illustrates folding of exemplary sectioned medical pad 500, according to some embodiments. In this example, foldable pad 500 again includes inner biocompatible hydrogel layer 340, fluid containing layer 350, and insulation layer 360. Due to the presence of perforations, seam, or segmenting structure 510, the user can fold extension section B without difficulty.

As described in the example of FIG. 6B, hydrogel layer 340, fluid containing layer 350, and/or insulation layer 360 may narrow close to perforations 510, where pad 500 is folded. In some embodiments, these layers may narrow more as the pad is folded, due to internal tension caused by the curvature of pad 500 in which the layers are contained. In some embodiments, this further narrowing may form a kink that constricts the fluid flow to extension section B (shown in FIGS. 6C-6D without fluid therein). In some embodiments, such a kink may form when pad 500 is folded, even if the pad and/or its internal layers are not narrowed when unfolded.

Additionally, as shown, the portions of hook and loop fasteners 610 that cover respective sections A and B of pad 500 may begin to meet as pad 500 is folded.

FIG. 6D illustrates an exemplary cross-sectional structure of folded medical pad 500, according to some embodiments. In this example, pad 500 again includes inner biocompatible hydrogel layer 340, fluid containing layer 350, and insulation layer 360.

As pad 500 is fully folded, the portions of hook and loop fasteners 610 that cover sections A and B of pad 500 may contact and fasten together, as shown, thereby stably attaching sections A and B together. In some embodiments, another method may be used to fasten folded sections A and B, for example an adhesive or cohesive material, or snaps.

When pad 500 is in a folded configuration, the TTM fluid may flow 620 through fluid containing layer 350 in main pad section A. However, the TTM fluid may be constricted by the fold or, e.g., a kink located at perforations, seam, or segmenting structure 510. In some embodiments, such a kink may naturally occur when perforations, seam, or segmenting structure 510 is folded. For example, as shown in FIG. 6D, hydrogel layer 340, fluid containing layer 350, and insulation layer 360 may narrow close to perforations 510, due to internal tension, as pad 500 is completely folded. In some embodiments, this further narrowing of fluid containing layer 350 may form a kink that constricts the fluid flow to extension section B. In some embodiments, such a kink may form when pad 500 is folded, regardless of whether or not pad 500 and/or its internal layers are narrowed near perforations 510 when pad 500 is in the unfolded state.

Alternatively, such a kink may be intentionally brought into place via a mechanism in pad 500, for example a valve, piston, drawstring, or lock. In various embodiments, such a mechanism may be user-operated (e.g., a drawstring may be tightened by a user), or be triggered automatically when pad 500 is folded.

Moreover, in some embodiments, the kink can help maintain a pressure differential between the folded and unfolded sections of the pad 500, due to the negative fluid pressure applied by the pump of the TTM system. In particular, the kink may be a sufficiently strong barrier to gaseous flow between the folded and unfolded portions of the pad, that it prevents the portion of fluid containing layer 350 in pad section B from being maintained by the TTM pump at the same negative pressure as section A. As a result, section B may be at a higher pressure than section A, particularly if the pad is folded before the TTM pump begins to operate. Thus, in addition to the kink directly constricting fluid flow into extension section B, TTM fluid may also be prevented from flowing into section B by the pressure differential. In this case, the flow 620 may be constrained especially effectively.

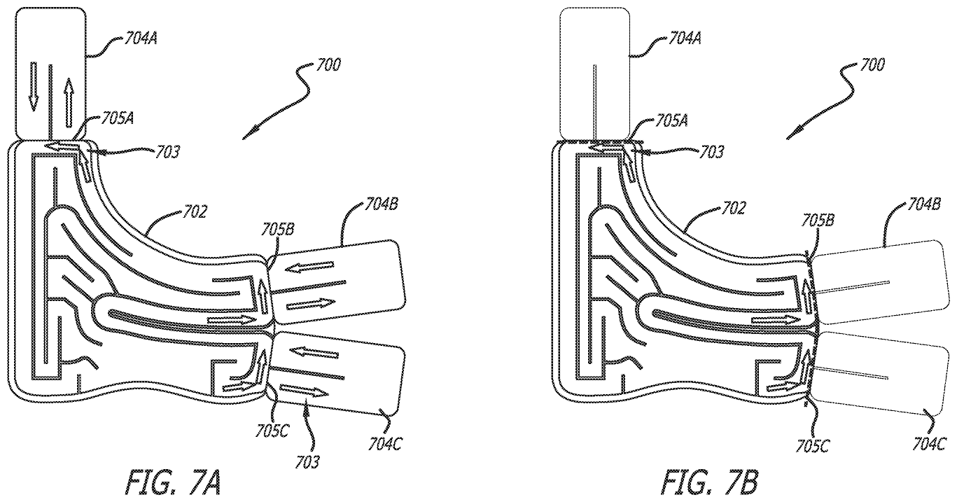

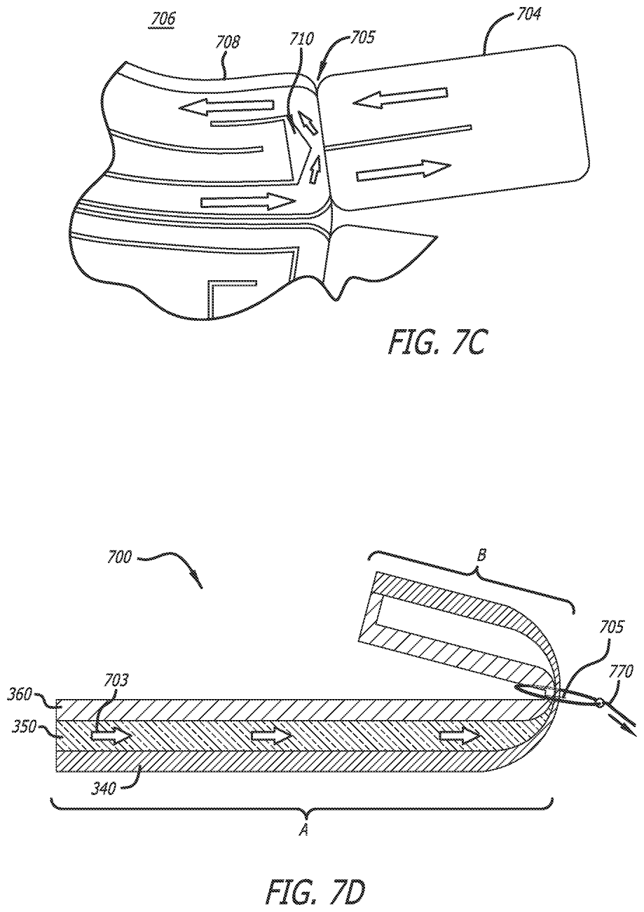

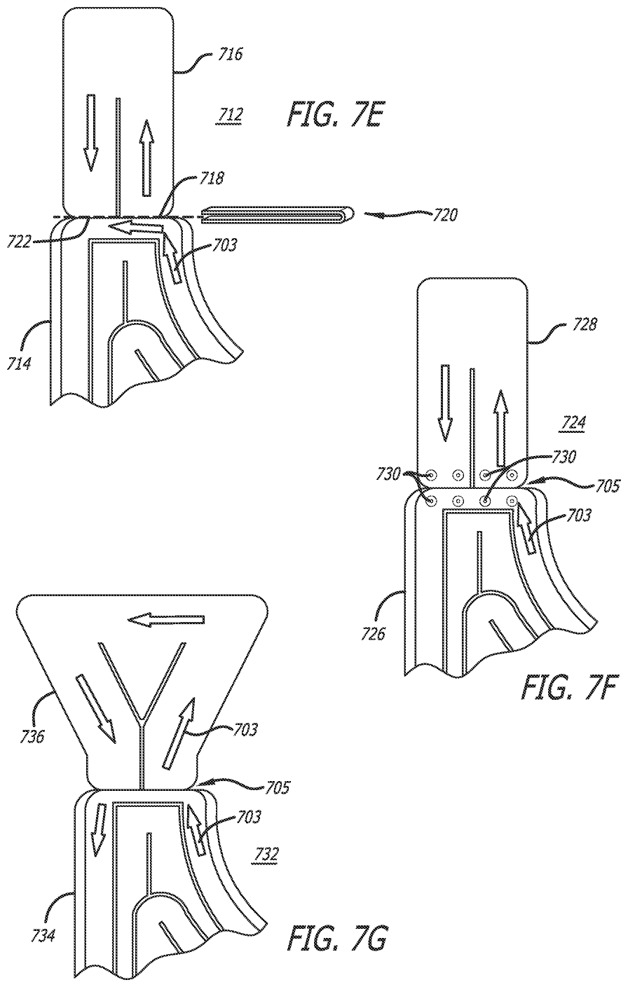

In some embodiments, the configuration of TTM fluid circulation lines 140 (see FIG. 1) is modified to accommodate the constricted fluid flow when pad 500 is folded. The TTM fluid circulation lines typically include an inlet line into the medical pad 500, and an outlet line from pad 500. In some embodiments, an adjustment is needed to this arrangement when flow 620 is constricted in the pad's folded configuration. Accordingly, in the example of FIG. 7B, a fluid inlet line can be the source of fluid flow 620 in main pad section A, while a secondary fluid outlet line can remove the fluid flow 620 exiting from main pad section A when pad 500 is in the folded configuration. Foldable medical pad 500 may also have a primary outlet line at an edge of extension section B for use when pad 500 is in the unfolded configuration (see FIG. 6A). The secondary outlet line can be located at an outer surface of main pad section A, which serves as a terminal edge of section A when pad 500 is in a folded configuration, as shown. When pad 500 is in an unfolded state, this same surface may be on a top or bottom of main pad section A of pad 500.

Alternatively, in another embodiment, both the inlet and outlet lines can be located at a first edge of main pad section A. In this case, the fluid flow 620 can reverse at a terminal edge of main pad section A when pad 500 is folded, flowing back to the outlet line at the first edge of section A.

Referring to now FIGS. 7A-7G, a plurality of embodiments of a medical pad including an extension are shown. In particular, some of the illustrations depict exemplary shapes of a medical pad and a corresponding extension; however, the intention of the various shapes is to provide for an understanding that the shape of the extension (or medical pad) is not limited to the specific shapes shown and that the disclosure should not be so limited. Additionally, some of the illustrations provide for specific user-operated closures such as a sliding closure, a cinch closure, a snap-fit closure, etc.

Referring to FIGS. 7A-7B, embodiments of a medical pad including foldable extensions are shown in an open state (FIG. 7A) and in a closed state (FIG. 7B), according to some embodiments. FIG. 7A illustrates a medical pad system 700 including a medical pad 702 and a plurality of extensions 704A-704C, wherein the embodiment illustrates three (3) extensions; however, the disclosure is not so limited. Instead, the medical pad system 700 may include an alternative number of extensions (e.g., one, two, four, etc.). A fold line 705A-705C is formed at the locations at which the medical pad 702 and the extensions 704A-704C are connected. As was discussed above, fluid 703 flows throughout the medical pad 702 and when an extension 704A-704C is in an open state, through the extension 704A-704C. Each of the extensions 704A-704C is shown in the open state in FIG. 7A such that the fluid 703 passes through a fluid path that extends between the medical pad 702 and the extensions 704A-704C.

FIG. 7B illustrates the medical paid system 700 of FIG. 7A with each of the extensions 704A-704C in a closed state, i.e., the fluid 703 does not, or substantially does not, pass through the fluid path between the medical pad 702 and the extensions 704A-704C but instead remains within the fluid containing layer of the medical pad 702 (such as the fluid containing layer 350 discussed above). Various embodiments of transitioning the extensions 704A-704C from the open state to the closed state (and vice versa). For example, as we illustrated above, the extensions 704A-704C may be folded at the fold lines 705A-705C, where the folding action creates a kink in the fluid path between the medical pad 702 and the extensions 704A-704C, as a result, the fluid 703 remains within the medical pad 702. Various embodiments illustrated in FIGS. 7C-7G provide further mechanisms and/or methods for securing the folds, i.e., to maintain the kink and prevent the fluid 703 from flowing through a kink into an extension 704A-704C.

Through the disclosure below, the extensions 704A-704C may be referred to individually as “extension 704” representing that such disclosure applies equally to any of the extensions 704A-704C. Similarly, the fold lines 705A-705C may be referred to individually as “fold line 705” representing that such disclosure applies equally to any of the fold lines 705A-705C.

Referring to now FIG. 7C, the medical pad system 706 is similar to the medical system 700 of FIGS. 7A-7B in that the medical pad 708 includes the same components as the medical pad 702 and includes the extension 704 such that fluid 703 is capable of flowing from the medical pad 708 to the extension 704 through a fluid path 705. The medical pad 708 differs from the medical pad 702 in that the medical pad 708 includes an angular divider 710 within the fluid containing layer 350, which promotes flow of the fluid 703 into the extension 704.

Referring now to FIG. 7D, a user-operated mechanism for forming a kink in a medical pad system 700 is shown according to some embodiments. In the example of FIG. 7D, the medical pad system 700 includes a drawstring 770 positioned at the fold line 705. Thus, the extension 704 may be folded at the fold line 705 to create a kink, which prevents or substantially prevents the fluid 703 from flowing into the extension 704. However, the drawstring 770, when tightened, further restricts the fluid flow. Accordingly, fluid flow 620 proceeds through the medical pad 702 while not entering into the extension 704.

Referring now to FIGS. 7E-7F, alternative embodiments of user-operated mechanisms for forming a kink in a medical pad are shown according to some embodiments. Referring to FIG. 7E, the medical pad system 712 includes a medical pad 714 that is similar to the medical pad 702 discussed above, and an extension 716 that is similar to the extension 704 also discussed above. Additionally, the medical pad system 712 includes the closure mechanism 718 that includes a sliding clip 720 and a sliding track 722, wherein the closure mechanism 718 is positioned at the fold line between the medical pad 714 and then extension 716. The sliding clip 720 functions to establish a kink and further block the fluid path between the medical pad 714 and the extension 716 when moved (slid) from a first position to a second position, where the second position includes the sliding clip 720 surrounding the exterior of the medical pad system 712 at the fold line.

Referring to FIG. 7F, the medical pad system 724 includes a medical pad 726 that is similar to the medical pad 702 discussed above, and an extension 728 that is similar to the extension 704 also discussed above. Additionally, the medical pad system 724 includes the snap closure mechanism 730 that operates to create and maintain a kink at the fold line 705. For example, when the medical pad system 724 is in the open state, the fluid 703 flows from the medical pay 726 into the extension 728 through a fluid path at the fold line. However, when the medical pad system 724 is placed in the closed state (e.g., folded), the snap closure mechanism 730 is snapped together such that a first component 732 mates with a second component 734. The mating of the first component 732 with the second component 734 maintains a kink formed via the fold at the fold line, thereby preventing, or substantially preventing, the fluid 703 from flowing into the extension 728.

Referring now to FIG. 7G, a medical pad having an alternatively-shaped extension is shown according to some embodiments. The medical pad system 732 includes a medical pad 734 that is similar to the medical pad 702 discussed above, and an extension 736 that is similar to the extension 704 also discussed above. The medical pad system 732 is intended to illustrate that the extension 736 need not be a particular shape, such as the rectangular shape of the extensions 704, 716, 728, etc., discussed above. Thus, it should be understood that the extension may be configured in various shapes in order to fit various body types and/or body parts.

In some examples, there are regions within the pad where the water flow rate is different than rest of the pads, which allows for controlled thermal energy transfer to patient body. For example, the extension 716 of FIG. 7E may be filled (completely or partially) prior to placement of the sliding clip 720, which either completely or substantially restricts the water flow between the extension 716 and the pad 714. For instance, the sliding clip 720 may substantially restrict the flow of water between the extension 716 and the pad 714 when the extension 716 is not folded over and completely restrict the flow of water therebetween in combination with the folding of the extension 716. Thus, when the sliding clip 720 is placed along the sliding track 722 (e.g., a designated area for placement of the clip 720), fluid 703 may enter the extension 716 at a flow rate less than the rate at which the fluid 703 travels within the pad 714. As a result, the temperature of the fluid 703 within the extension 716 may be at a different temperature than the fluid 703 within the pad 714. Such an embodiment may be advantageous when a clinician desires for an extremity or other portion of a patient to remain warm but not rise to the temperature of the fluid 703 within the pad 714 when the TTM procedure is providing a heating effect (or alternatively, remain cool but not dip to the temperature of the fluid within the pad 714 when the TTM procedure is providing a cooling effect).

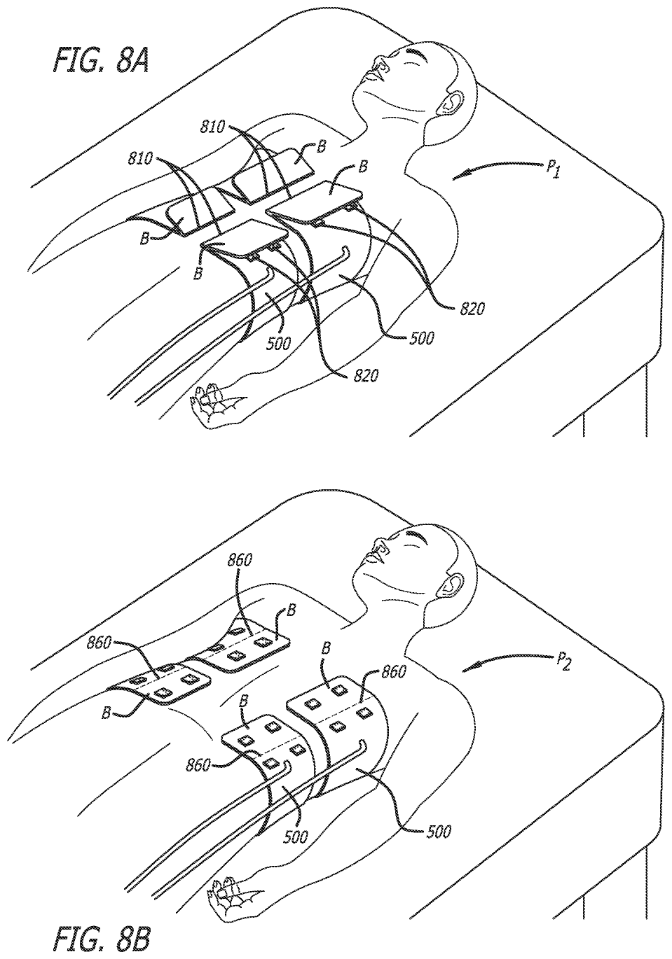

FIG. 8A shows usage of the medical pads 500 with folded extension sections B, according to some embodiments, on the patient P₁ of FIG. 4A. In this example, if unfolded, pads 500 would be too large for patient P₁, as shown in the example of FIG. 4A. Consequently, the extension sections B are folded, thereby reducing the contact area of pads 500 on patient P¹. Folded TTM pads 500 are effectively reduced to an appropriate size for patient P₁, without overlapping, and cover much of the back and chest of patient P₁ while leaving some areas uncovered. Moreover, the appropriate fit of folded pads 500 enables pads 500 to heat or cool patient P₁ efficiently and effectively.

The pads 500 may be folded along perforations, seam, or segmenting structure 810, as described above (see FIG. 7A), thereby forming a constriction or kink that may constrict flow of the TTM fluid (see FIG. 7B). Accordingly, no TTM fluid may flow in extension sections B, thereby reducing wasted energy. In this example, pads with hook and loop fasteners 820 can also maintain extension sections B in a stable position when folded.

FIG. 8B shows usage of the medical pads 500 with unfolded extension sections B, according to some embodiments, on the patient P₂ of FIG. 4B. In this example, if folded, pads 500 would be too small for patient P₂, similar to the undersized pads shown in the example of FIG. 4B. Consequently, a user, such as a clinician, can unfold extension sections B, thereby expanding the contact area of pads 500 making contact with patient P₂. In the unfolded configuration, the pads 500 can circulate a greater quantity of TTM fluid than when folded, thereby heating or cooling patient P₂ effectively.

In this example, extension sections B are unfolded along perforations, seam, or segmenting structure 860, and therefore can conform to the contours of patient P₂. Moreover, the TTM fluid can flow throughout the pad 500 with an enlarged patient contact surface corresponding to both the main section and section B, with no obstruction or reduction of flow. As a result, unfolded pad 500 is large enough to heat or cool patient P₂ effectively.

In some examples, a patient may be of a medium size intermediate between patients P₁ and P₂, and therefore the clinician may choose to fold a subset, but not all, of extension pads B.

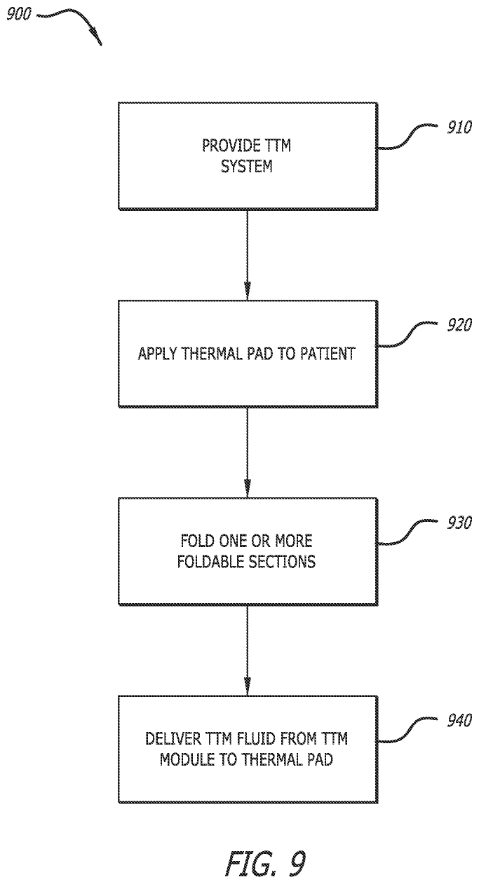

FIG. 9 shows a flowchart of a method 900 for providing a TTM therapy to a patient, according to some embodiments. Each block illustrated in FIG. 9 represents an operation performed in the method 900 of providing a TTM therapy to a patient. In various embodiments, the method can be performed by one or more users, such as nurses, doctors, or other clinicians, etc.

As an initial step in the method 900, the user can provide a TTM system comprising a fluid containing layer for containing the TTM fluid and a patient contact surface (block 910). The fluid containing layer is configured for circulating the TTM fluid within the fluid containing layer. The patient contact surface defines a patient contact area to facilitate thermal energy exchange with the patient. The pad is segmented into a main pad section and one or more foldable sections.

Next, the user can apply the thermal pad to the patient (block 920). As described in the example of FIG. 2, the user can align the top of a first upper body pad with axilla of the patient's outstretched arm. The user can then place the pad along the side of the patient's spine. The user can wrap the pad from the patient's back to front. The user can align the first lower body pad's lines with the knee and point downward. The user can wrap the first lower body pad laterally, and overlap medially if needed. The user may then turn the patient and place a second upper body pad on the patient's other side, leaving a space along the patient's spine. The user can wrap a second lower body pad around the patient's other leg. Finally, if additional surface coverage is needed, the user can optionally place a universal medical pad on the patient's abdomen.

As a next step in the method 900, the user can fold the one or more foldable sections (block 930), thereby reducing the patient contact area of the thermal pad. Accordingly, the contact area of the pad is effectively reduced to the contact area of the main pad section, as well as any extension sections that remain unfolded. The folded thermal pad can effectively be reduced to an appropriate size for the patient, and can cover much of the patient's body. Moreover, this appropriate fit enables the folded thermal pads to heat or cool the patient efficiently and effectively.

Alternatively, in some embodiments, the user can unfold one or more folded sections of the thermal pad, thereby effectively increasing the patient contact area. In the unfolded configuration, the thermal pad can circulate a greater quantity of TTM fluid than when folded, thereby heating or cooling the patient effectively.

Finally, the user can configure the TTM system to deliver TTM fluid from the TTM module to the thermal pad via the fluid delivery line (FDL) (block 940). As described above, for example in regard to FIG. 7B, when extensions B and C are folded, a kink may constrict fluid flow into extensions B and C, so that TTM fluid flows only in main pad section A. In this case, the contact area of the pad is effectively reduced to main section A. Alternatively, if any of the extension sections remains unfolded, the TTM fluid may flow normally in those sections.

In some embodiments, the kink may naturally form when the thermal pad is folded. Alternatively, such a kink may be intentionally brought into place via a mechanism in the thermal pad, for example a valve, piston, drawstring, or lock.

In some embodiments, the configuration of TTM fluid circulation lines is adjusted to accommodate the constricted fluid flow when pad 500 is folded. For example, a fluid inlet line can be the source of fluid flow in the main pad section, while a secondary fluid outlet line can remove the fluid flow exiting from main pad section when pad is in the folded configuration (see FIG. 7B).

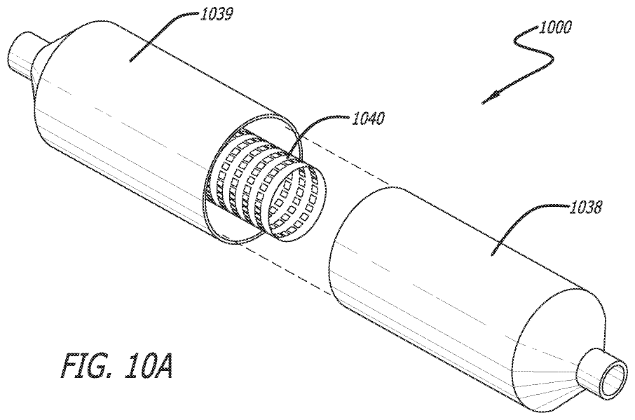

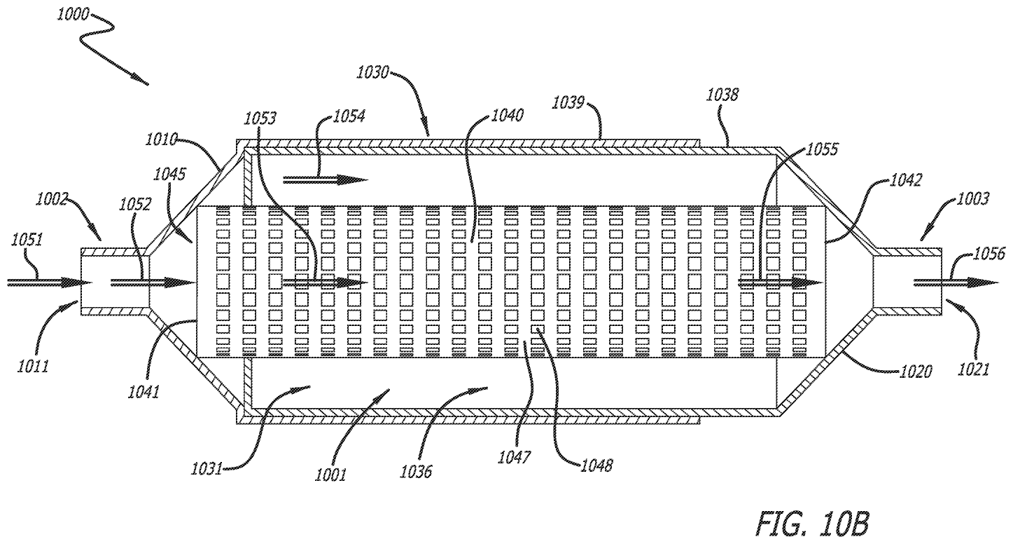

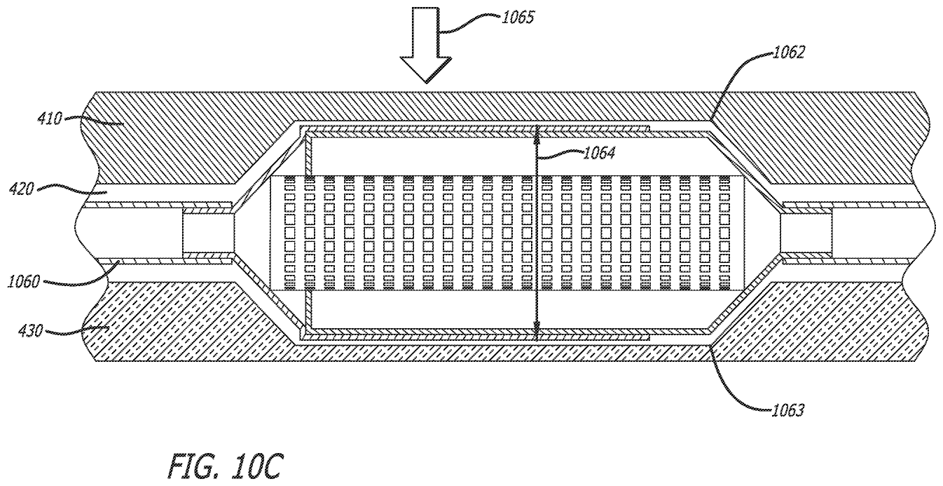

FIGS. 10A and 10B show a filter 1000 that may be included with the TTM system 100. The filter 1000 may be disposed in line with a TTM fluid flow path of the TTM system 100 so that the circulating TTM fluid 112 flows through the filter 1000. The filter 1000 may be configured to remove (i.e., filter out) material/particles having a size of 0.2 microns or larger from the TTM fluid 112 without causing a flow restriction of the TTM fluid 112.

The filter 1000 comprises a longitudinal shape having a flow path 1001 extending from a first end 1002 to a second end 1003. The filter 1000 comprises a diffuser 1010 adjacent the first end 1002, a nozzle adjacent 1020 the second end 1003, and a body 1030 extending between the diffuser 1010 and the nozzle 1020. Along the diffuser 1010, a cross-sectional flow area of the filter 1000 expands from an inlet flow area 1011 to a body flow area 1031 and along the nozzle 1020, the cross-sectional flow area of the filter 1000 contracts from the body flow area 1031 to an outlet flow area 1021. In some embodiments, the inlet flow area 1011 and the outlet flow area 1021 may be substantially equal.

In some embodiments, the body flow area 1031 may be constant along the body 1030. In other embodiments, the body flow area 1031 may vary along a length of the body 1030 such that the body flow area 1031 is greater or less along middle portion of the body 1030 than at the ends of the body 1030. In some embodiments, the body flow area 1031 may be circular.

The filter 1000 comprises an inner tube 1040 disposed within the body 1030 extending along the length of body 1030. The inner tube 1040 may be coupled to the diffuser 1010 at a first inner tube end 1041 so that TTM fluid 112 entering the filter 1000 at the first end 1002 also enters the inner tube 1040 at the first inner tube end 1041. The inner tube 1040 may be coupled to the nozzle 1020 at a second inner tube end 1042 so that TTM fluid 112 exiting the filter 1000 at the second end 1003 also exits the inner tube 1040 at the second inner tube end 1042.

The inner tube 1040 comprises an inner tube flow area 1045 extending the length of the inner tube 1040. The inner tube flow area 1045 may be greater than the inlet flow area 1011 and/or the outlet flow area 1021. The inner tube flow area 1045 may be constant along the length of the inner tube 1040. In some embodiments, the inner tube flow area 1045 may vary along the length of the inner tube 1040. In some embodiments, the inner tube 1040 may comprise a circular cross section. The inner tube 1040 and the body 1030 may be configured so that the body flow area 1031 comprises a combination of the inner tube flow area 1045 and an annular flow area 1036.

The inner tube 1040 comprises a porous a circumferential wall 1047. The porous wall 1047 may be configured so that TTM fluid 112 may flow through the porous wall 1047, i.e., through the pores 1048 of the porous wall 1047. Consequently, TTM fluid 112 may flow through the porous wall 1047 from the inner tube flow area 1045 to the annular flow area 1036 and from the annular flow area 1036 into the inner tube flow area 1045.

In use, the longitudinal velocity of the TTM fluid 112 may change along the length of the filter 1000. As the volumetric TTM fluid 112 flow through the filter is constant, the longitudinal velocity of the TTM fluid 112 may be at least partially defined by the flow areas of the filter 1000 as described below. The TTM fluid 112 may enter the filter 1000 at a first longitudinal velocity 1051 and decrease along the diffuser so that the TTM fluid 112 enters the inner tube at a second velocity 1052 less than the first longitudinal velocity 1051. At this point, a portion of the TTM fluid 112 may flow through the porous wall 1047 from the inner tube flow area 1045 into the annular flow area 1036 to divide the fluid flow into a third velocity 1053 within the inner tube flow area 1045 and a fourth velocity 1054 within the annular flow area 1036. The fourth velocity 1054 may be less than the third velocity 1053. A portion of the TTM fluid 112 may then flow back into the inner tube flow area 1045 from the annular flow area 1036 to define a fifth velocity 1055 along the inner tube flow area 1045 which may be about equal to the second velocity 1052. The TTM fluid 112 may then proceed along the nozzle 1020 to define a sixth velocity 1056 exiting the filter 1000. In some embodiments, the first velocity 1051 and the sixth velocity 1056 may be about equal.

The filter 1000 may be configured to remove harmful bacteria and viruses from the TTM fluid 112 using sedimentation principles. In use, the filter 1000 may be oriented horizontally so that the direction of fluid flow through the filter 1000 is perpendicular to a gravitational force 1065. In some instances, bacteria, viruses, and other particles within the TTM fluid 112 may have a greater density than the TTM fluid 112 and as such may be urged by the gravitational force 1065 (i.e., sink) in a direction perpendicular to the fluid flow direction. In some instances, particles within the inner tube flow area 1045 may sink toward and through the porous wall 1047 into the annular flow area 1036. Particles within the annular flow area 1036 may then sink toward an inside surface 1031 of the body 1030 and become trapped adjacent the inside surface 1031. The geometry of the filter 1000 may be configured to allow 0.2-micron bacteria/virus particles to fall out of the flow of TTM fluid 112 and become trapped along the inside surface 1031.

In some embodiments, the filter 1000 may be configured so that flow of TTM fluid 112 from the inner tube flow area 1045 into the annual flow area 1036 my drag particles through the porous wall 1047. In some embodiments, the inlet flow area 1011, the inner tube flow area 1045, and the annual flow area 1036 may be sized so that the third velocity 1053 is less than about 50 percent, 25 percent, or 10 percent of the first velocity 1051 or less. In some embodiments, the body 1030 and the inner tube 1040 may be configured so that the fourth velocity 1054 is less than the third velocity 1053. In some embodiments, the fourth velocity 1054 may less than about 50 percent, 25 percent, or 10 percent of the third velocity 1053 or less.

In some embodiments, the filter 1000 may be configured so that the flow within the inner tube flow area 1045 is laminar flow, i.e., so that the velocity of the fluid flow adjacent to or in close proximity to an inside surface 1041 of the porous wall 1047 is less than the velocity at a location spaced away from the inside surface 1041. In such an embodiment, the particles may more readily sink toward and through the porous wall 1047.

In some embodiments, the filter 1000 may be configured so that the fluid flow within the annual flow area 1036 is laminar flow, i.e., so that the velocity of the fluid flow adjacent to or in close proximity to inside surface 1031 of the body 1030 is less than the velocity at a location spaced away from the inside surface 1031. In such an embodiment, the particles may more readily sink toward and be trapped along the inside surface 1031.

The filter 1000 may comprise three components including the inner tube 1040 an inner body shell 1038, and an outer body shell 1039. Each of the three components may be formed via the plastic injection molding process. Assembly of the filter 1000 may include capturing the inner tube 1040 within the inner body shell 1038 and the outer body shell 1039 and sliding the inner body shell 1038 into the outer body shell 1039 wherein the fit between the inner body shell 1038 and the outer body shell 1039 is an interference fit.

In some embodiments, the filter 1000 may be disposed within the pad assembly 120. FIG. 10C shows a detail cross-sectional view of the pad assembly 120 including the filter 1000 disposed within the fluid containing layer 420. The filter 1000 is coupled in line with an internal flow path 1060 within the fluid containing layer 420 so that TTM fluid 12 circulating within the pad assembly 120 passes through the filter 1000. The filter 1000 may be sized so that the inlet flow area 1011 and the outlet flow area 1021 are similar to a cross-sectional flow area of the internal flow path 1060 within the fluid containing layer 420.

In some embodiments, a thickness of the fluid containing layer 420 may increase adjacent the filter 1000 to accommodate a body diameter 1064 of the filter 1000. To further accommodate the body diameter 1064, the insulation layer 410 and/or the thermal conduction layer 430 may comprise internal depressions 1062, 1063, respectively.

In some embodiments, one or more filters 1000 may be disposed in line with the flow of TTM fluid 112 at other locations of the TTM system 100. In some embodiments, one or more filters 1000 may be disposed within the TTM module 110. In some embodiments, one or more filters 1000 may be disposed in line with the FDL 130. In some embodiments, the filter 1000 may be disposed in line with a fluid conduit of the pad external to the fluid containing layer 420 such as a conduit extending between the pad connector 652 and the pad assembly 120.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical pad for exchanging thermal energy between a targeted temperature management (TTM) fluid and a patient, the pad comprising:
a fluid containing layer for containing the TTM fluid, the fluid containing layer configured for circulating the TTM fluid within the fluid containing layer; and
a patient contact surface defining a patient contact area to facilitate thermal energy exchange with the patient, wherein
the pad is segmented into a main pad section and one or more foldable sections, wherein the one or more foldable sections are each configured to be folded at a crease by a user, thereby reducing the patient contact area.

2. The medical pad of claim 1, wherein the circulation of the TTM fluid within the fluid containing layer is constricted by a fold of the one or more foldable sections while the one or more foldable sections are folded.

3. The medical pad of claim 2, wherein the TTM fluid does not circulate in the one or more foldable sections while folded.

4. The medical pad of claim 2, wherein the fold comprises a kink that constricts the circulation.

5. The medical pad of claim 1, wherein the pad is segmented by means of perforation or holes disposed through a thickness of the pad.

6. The medical pad of claim 5, wherein the pad is configured to be folded along the perforation or holes.

7. The medical pad of claim 5, wherein the thickness of the pad narrows in a vicinity of the perforation or holes.

8. The medical pad of claim 7, wherein the fluid containing layer narrows within the pad in the vicinity of the perforation or holes.

9. The medical pad of claim 1, further comprising hook and loop fasteners, the hook and loop fasteners configured to secure the one or more foldable sections to the main pad section while the one or more foldable sections are folded.

10. The medical pad of claim 1, wherein the one or more foldable sections are separately foldable.

11. The medical pad of claim 1, wherein the patient contact surface conforms to a body of the patient.

12. The medical pad of claim 1, further comprising a sliding clip, wherein the sliding clip is configured for placement at the crease to restrict flow of TTM fluid into a first foldable section.

13. The medical pad of claim 12, wherein the first foldable section is unfolded and the sliding clip is placed at the crease, a flow rate of the TTM fluid into the first foldable section is less than a flow rate of the TTM fluid within the main pad section.

14. The medical pad of claim 1, wherein the main pad section includes an angular divider within the fluid containing layer, wherein the angular divider promotes flow of the TTM fluid into a first foldable section when the first foldable section is unfolded.

15. The medical pad of claim 1, wherein the main pad section has a shape of one of a circle, an oval, a rectangle, or a “L” shape.

16. A method of providing a targeted temperature management (TTM) therapy to a patient, comprising:
providing a TTM system comprising:
a TTM module configured to provide a TTM fluid;
a thermal pad configured to receive the TTM fluid from the TTM module to facilitate thermal energy transfer between the TTM fluid and the patient; and
a fluid delivery line (FDL) extending between the TTM module and the thermal pad, the FDL configured to provide TTM fluid flow between the TTM module and the thermal pad, wherein
the thermal pad comprises a patient contact surface defining a patient contact area to facilitate thermal energy exchange with the patient, and
the thermal pad is segmented into a main pad section and one or more foldable sections;
applying the thermal pad to the patient;
folding the one or more foldable sections at a crease, thereby reducing the patient contact area of the thermal pad; and
delivering TTM fluid from the TTM module to the thermal pad via the FDL.

17. The method of claim 16, wherein:
the thermal pad further comprises a fluid containing layer for containing the TTM fluid, the fluid containing layer configured for circulating the TTM fluid within the fluid containing layer, and
the circulation of the TTM fluid within the fluid containing layer is constricted by a fold of the one or more foldable sections while folded.

18. The method of claim 17, wherein the TTM fluid does not circulate in the one or more foldable sections while folded.

19. The method of claim 17, wherein the fold comprises a kink that constricts the circulation.

20. The method of claim 16, wherein the pad is segmented by means of perforation or holes disposed through a thickness of the pad.

21. The method of claim 20, wherein folding the one or more foldable sections comprises folding along the perforation or holes.

22. The method of claim 20, wherein the thickness of the pad narrows in a vicinity of the perforation or holes.

23. The method of claim 22, wherein:
the thermal pad further comprises a fluid containing layer for containing the TTM fluid, the fluid containing layer configured for circulating the TTM fluid within the fluid containing layer, and
the fluid containing layer narrows within the pad in the vicinity of the perforation or holes.

24. The method of claim 16, wherein the thermal pad further comprises hook and loop fasteners, the hook and loop fasteners configured to secure the one or more foldable sections to the main pad section while the one or more foldable sections are folded.

25. The method of claim 16, wherein folding the one or more foldable sections comprises separately folding a subset of the one or more foldable sections.

26. The method of claim 16, wherein the patient contact surface conforms to skin of the patient.

27. The method of claim 16, further comprising unfolding the one or more foldable sections.

28. The method of claim 16, wherein a sliding clip placed at the crease to restrict flow of TTM fluid into a first foldable section.

29. The method of claim 28, wherein the first foldable section is unfolded and the sliding clip is placed at the crease, a flow rate of the TTM fluid into the first foldable section is less than a flow rate of the TTM fluid within the main pad section.

30. The method of claim 16, wherein the main pad section includes an angular divider within the fluid containing layer, wherein the angular divider promotes flow of the TTM fluid into a first foldable section when the first foldable section is unfolded.

31. The method of claim 16, wherein the main pad section has a shape of one of a circle, an oval, a rectangle, or a “L” shape.
